(19) United States
(12) Patent Application Publication
(10) Pub. No.: US 2024/0241019 A1
(43) Pub. Date: Jul. 18, 2024

(54) DEVICE AND METHOD FOR TISSUE STAINING QUALITY CONTROL

(71) Applicant: FUTAMURA CHEMICAL UK LTD, Wigton (GB)

(72) Inventors: Martin Richard COCKROFT, Wigton (GB); Elizabeth KEATING, Wigton (GB)

(21) Appl. No.: 18/559,250

(22) PCT Filed: Apr. 27, 2022
(86) PCT No.: PCT/EP2022/061268

§ 371 Date: Nov. 6, 2023

(30) Foreign Application Priority Data
May 6, 2021 (GB) 2106487.8

(51) Int. Cl. G01N 1/30; C07K 14/47; C08B 37/08; C08L 1/02; G06T 7/00

(52) U.S. Cl. CPC G01N 1/30; C07K 14/4741; C08B 37/003; C08L 1/02; G06T 7/0012; G06T 2207/30024

(57) ABSTRACT

Disclosed herein are quality control devices and methods for histopathology staining. Specifically disclosed is a device that includes a cellulose film containing less than two plasticizers.

This application is a National Phase entry of International Application No. PCT/EP2022/061268 under § 371 and claims the benefit of GB Patent Application No. 2106487.8, filed May 6, 2021, which is hereby incorporated by reference in its entirety.

### FIELD OF THE DISCLOSURE

The present disclosure relates to quality control devices and methods for histopathology staining.

### BACKGROUND

Histopathology relies on the use of a range of complex staining processes to allow the visualization of cells and tissue within a sample. However, there is a known degree of variance in such staining processes, depending on the stains and the process conditions used. For example, stain time, stain concentration and tissue thickness can all influence the resulting stained image.

Digital imaging in histopathology is becoming increasingly important in the medical profession, but digitizing these images adds further complexity due to variations in the imaging process, such as changes in light intensity or the color response of the camera used. These variations are at risk of reducing the credence that can be given to digital pathology.

If the variance arising during the staining and imaging processes due to the variations in staining and imaging conditions can be quantified, the resulting image can be corrected to account for this variance. This ensures that the resulting digitally processed images contain the minimum possible degree of variance, thereby calibrating the images and improving their quality.

Thus, there a requirement for a way to quantify the variance arising during a histopathological staining process and the subsequent imaging process. This could be achieved using a quality control device, as is discussed in GB2522231. This document discloses a stain assessment target comprising a substrate formed from an optically transmissive material that is analogous to biological tissue. This substrate is stained under similar conditions as the tissue sample and thereby provides a calibration device for comparing the resulting images.

Similarly, WO2013/186530 discloses an imaging calibration device in which a stain is applied to a region of a surface, which may include individual tissue blocks. These stained regions can then be used to calibrate the resulting images.

Any such quality control device must take up a stain with low variability, such that it can provide a reliable comparison for quantifying the variance in staining and imaging processes. This is discussed in GB2524227, which discloses an imaging reference device having a first region of a first substrate and a second region of a second substrate, wherein the regions are stained with different tissue stains and then overlapped. This produces improved reference devices, which can closely match the spectral characteristics of the tissue sample. However, this is a more complex system and requires a separate device to be created for each staining protocol.

Thus, there is required a quality control device for histopathology staining that shows low variability in stain uptake but is simple and easy to use.

### SUMMARY

It has been discovered that cellulose films provide good stain uptake when used as a quality control device for histopathology staining and imaging. The cellulose film can be stained at the same time and under the same conditions as the tissue sample of interest and is stained proportionately to the tissue sample using a variety of stains including hematoxylin and eosin (H&E), or diaminobenzidine (DAB) and horseradish peroxide (hrp).

The cellulose film therefore provides an internal color control, which allows the characterization of color changes during sectioning, staining and imaging. This allows independent measurement of the illumination, the imaging device and the individual stains applied to the slide, thereby allowing the variation arising from the staining and imaging processes to be quantified and corrected, as shown in FIG. 1.

A quantitative assessment of the tissue staining process and any subsequent imaging process is then possible, which can reduce interobserver variation and allow digitization of the staining results. This quantitative assessment allows the digitally processed images to be corrected to account for any variations, thereby improving the quality of the collected images.

According to a first aspect of the present invention, there is provided a quality control device for histopathology staining comprising a cellulose film containing less than two (i.e. one or zero) plasticizers.

Cellulose films generally have complex plasticizer packages containing two or more different plasticizers. This allows the properties of the resulting films to be tailored to the technical requirements of different applications, and also has commercial implications, such as balancing fluctuations in cost or supply. For example, plasticizers are generally considered essential to reduce the brittleness of conventional cellulose films.

It has surprisingly been found that cellulose films with a single plasticizer or no plasticizer (i.e. less than two different plasticizers) showed a reduced variability in stain uptake, thereby creating a more consistent product. Without wishing to be bound by theory, it is thought that cellulose films having two or more plasticizers have increased sources of variation in staining. The predictability of stain uptake is important in a quality control device in order to provide a reproducible comparative measurement.

One of the additional challenges of using a cellulose film as a quality control reference material is holding it in place on a slide surface throughout the course of the slide preparation such that it remains intact, flat and suitable for imaging. This is because the dimensions of the cellulose film change in different hydration states, meaning that a cellulose film can undergo a range of dimensional changes that are strong enough to cause detachment from the slide or another surface to which the film is attached.

It was surprisingly found that simpler plasticizer packages (i.e. containing one plasticizer) or the absence of a plasticizer helped to reduce lifting of the cellulose film from a surface such as a slide, to which the film was adhered. Without wishing to be bound by theory, it is thought that the plasticizer may leach out of the cellulose film and into the adhesive between the film and the surface, thereby destroying its integrity and allowing for liquid ingress behind the film. This leaching can further increase the image variability arising during the staining and imaging processes.

It was also found that the resulting increased brittleness of the cellulose film was not problematic in the quality control device of at least one embodiment of the present invention due to the low stress profile of such devices, particularly when attached to a surface or when comprising a protective sheet.

The variability of stain uptake increased with increasing number of different plasticizers present in the cellulose film. The most predictable stain uptake was seen with a cellulose film that is free from plasticizers.

Plasticizers are well known for use in cellulose films and any conventional plasticizer may be used in the cellulose film of at least one embodiment of the present invention. Suitable plasticizers include glycerol, polyethylene glycol, monopropylene glycol, triethylene glycol and urea.

The cellulose film may comprise one or more reference articles. Reference articles are particles that are visible within the film following the staining process, thereby allowing size comparisons to be made with articles found in the tissue sample and enabling easier focusing of the image. Reference articles can also be used to visually represent the tissue itself, to aid the pathologist in reviewing the sample.

Reference articles are preferably of a known size, orientation and/or concentration within the cellulose film. The reference articles are preferably embedded within the cellulose film. The reference articles may have been embedded in the film during its manufacturing.

The reference articles may comprise inert particles, such as polymethyl methacrylate (PMMA) or silica particles. The inert particles may be of a known particle size. The inert particles may be of varying particle size with a known range of sizes. The inert particles may be of a known concentration within the cellulose film.

The reference articles may comprise cellulose fibers. The cellulose fibers may be of known fiber length and/or diameter. The cellulose fibers may be of known orientation and/or of a known concentration within the cellulose film.

The reference articles may comprise air bubbles. The air bubbles may be of controlled and/or known size. The air bubbles may be of known concentration within the cellulose film.

The reference articles may comprise proteins. The proteins may be those found in the tissue sample of interest, such as her-2 and estrogen receptor proteins. The concentration of the proteins in the cellulose film may correspond to the concentration of proteins expected in the tissue sample of interest. The proteins may be of known size and/or of a known concentration within the cellulose film.

The cellulose film may comprise a combination of different reference articles. The reference articles may be used to replicate one or more characteristics of the tissue sample of interest.

Thus, the reference articles may have similar properties, such as size and/or staining color, as particles of interest within the tissue sample. A variety of reference particles having a range of values for a particular property, such as size or staining color, may be included in the film to offer a fast reference when imaging.

The reference articles may have a random distribution within the film. Thus, the pattern of the reference articles can be used to generate a unique “digital fingerprint” for each quality control device, allowing traceability of the device through its history.

The cellulose film may comprise chitosan. The chitosan may be fungal chitosan. This can reduce stain uptake variability. The variability of staining decreases with increasing chitosan levels in the film. The film may comprise chitosan at 1 to 10%, preferably 3 to 10% and most preferably 5 to 10%.

The cellulose film may comprise dopants that aid the stain uptake of the cellulose film. Different tissues have different degrees of stain uptake, particularly with stains such as eosin and hematoxylin. These dopants can be included in an amount such that the stain uptake of the cellulose film matches the stain uptake of the tissue sample of interest. Thus, the cellulose film can be tailored to the tissue sample of interest.

Both keratin and gelatin have been found to increase stain uptake in the cellulose film. Chitosan also increases the stain uptake of the cellulose film. Thus, the cellulose film may comprise one or more of chitosan, keratin and gelatin in order to tailor the stain uptake of the cellulose film to mirror the staining of the tissue sample of interest.

The cellulose film may be between 10 and 50 μm thick. The cellulose film may be between 20 and 30 μm thick. This thickness helps to reduce the change in dimensions of the film due to different hydration states, which can reduce lifting of the film from a surface such as a slide, thereby reducing stain uptake variation.

The degree of variation in cellulose film thickness is preferably low, as this helps to further decrease the variability in stain uptake of the cellulose film. The thickness variation may be less than 3% of its target value, preferably less than 2%.

The quality control device may comprise pre-colored red, blue and green regions. This can improve accuracy when quantifying the variation resulting from the staining and imaging processes by providing color standards on the device.

The cellulose film may have an adhesive on at least one side thereof. The cellulose film may have an adhesive on both sides thereof.

The cellulose film may be adhered to a protective sheet. The cellulose film may be laminated to a protective sheet. This can reduce the change in dimensions of the film due to different hydration states, thereby reducing stain uptake variation. The protective sheet can also protect the cellulose film from damage during use as a quality control device.

The protective sheet may comprise a polymer. The protective sheet may be a film.

The quality control device may further comprise an adhesive on the opposite side of the cellulose film to the protective sheet. This allows the quality control device to be attached to the same surface as the tissue to be stained, thereby ensuring that the tissue and the quality control device are subjected to the same staining conditions. The surface may be a microscope slide.

The cellulose film may comprise a coating on at least one side thereof. The cellulose film may also comprise a primer layer between the cellulose film and the coating. The cellulose film may be absent a coating and/or a primer layer. If present, the coating is preferably on the side of the film adjacent the surface, opposite the protective sheet (if present), such that the stain is not prevented from reaching the cellulose film. An adhesive may be applied over the coating, in order to adhere the cellulose film to a surface.

The coating may be a barrier coating. This can prevent the plasticizer from leaching into the adhesive, thereby destroying its integrity and reducing the adhesion between the film and the surface. The coating may also increase the adhesion between the adhesive and the cellulose film itself. The coating may comprise ethylene acrylic acid, polyvinylidene chloride, acrylic or any other barrier coating material.

According to a second aspect of the present invention, there is provided a surface comprising the quality control device discussed above adhered thereto. The surface may be a surface of a microscope slide.

The quality control device may not cover the entire surface. A tissue sample can then be positioned on the surface and stained simultaneously with the quality control device, thereby ensuring that the tissue sample and the quality control device are subjected to identical staining conditions.

According to a third aspect of the present invention, there is provided a use of a cellulose film containing less than two plasticizers in a quality control device for histopathology staining.

As discussed above, the presence of either one or zero plasticizers reduces stain uptake variability in the cellulose film, thereby creating a more consistent quality control device for histopathology staining.

According to a fourth aspect of the present invention, there is provided a use of a quality control device discussed herein for correcting image variation in a histopathological staining and imaging process.

This is possible due to the predictable and linear stain uptake of the cellulose film of at least one embodiment of the present invention, which creates a reliable color control that can be used to quantify the variation arising from the staining and imaging process, thereby allowing correction of said variation in the resulting images.

According to a fifth aspect of the present invention, there is provided a method of correcting image variation arising from a process of staining and imaging a tissue sample comprising:
staining a quality control device discussed above and the tissue sample under the same conditions;
imaging the quality control device;
quantifying the staining and imaging variation in the quality control device image;
imaging the tissue sample; and
correcting the tissue sample image using the quantification of the staining and imaging variation in the quality control device image.

The quality control device and the tissue sample may be stained simultaneously, under the same staining conditions at the same time. Preferably, the quality control device and the tissue sample are attached to the same surface, such as a microscope slide, and are stained together as this ensures that they are subjected to the same staining conditions.

The steps of this method can be conducted in a different order to that outlined above. For example, imaging the tissue sample may occur before the staining and imaging variation in the quality control device image is quantified.

### BRIEF DESCRIPTION OF THE DRAWINGS

The invention will now be more particularly described with reference to the following examples and figures, in which:

FIG. 1 illustrates the process of correcting an image of a stained tissue sample using a quality control device, according to an embodiment of the present invention;

FIG. 2 illustrates the linearity of the stain uptake in zero-plasticizer cellulose films, according to an embodiment of the present invention;

FIG. 3 illustrates the linearity of the stain uptake in single-plasticizer cellulose films, according to an embodiment of the present invention; and

FIG. 4 illustrates the eosin uptake of single-plasticizer cellulose films according to embodiments of the present invention having different chitosan doses.

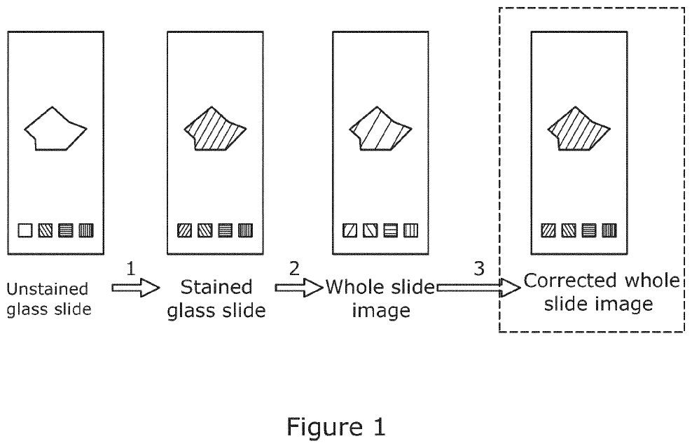

### DETAILED DESCRIPTION

### Example 1

The change in stain intensity with increasing stain time was investigated using an uncoated cellulose film comprising one plasticizer and an uncoated cellulose film without any plasticizer.

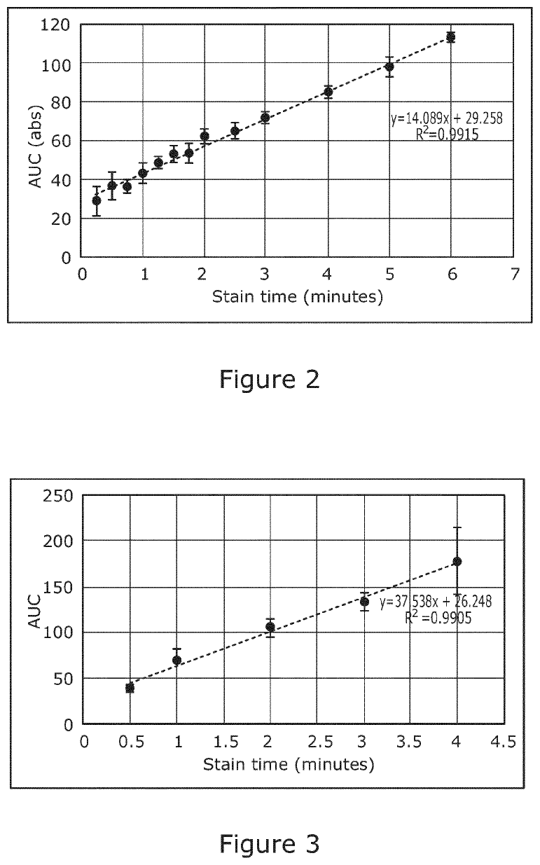

FIG. 2 illustrates the increase in stain intensity with increasing stain time for the zero-plasticizer cellulose film, while FIG. 3 illustrates the increase in stain intensity with increasing stain time for the single-plasticizer cellulose film.

The R² value for a cellulose film without plasticizers is 0.9915, while the R² value for a single-plasticizer cellulose film drops to 0.9905. These graphs demonstrate that the addition of a plasticizer causes increased variability in the stain uptake of the cellulose film of at least one embodiment of the invention. However, this variability is statistically acceptable in both cellulose films tested.

### Example 2

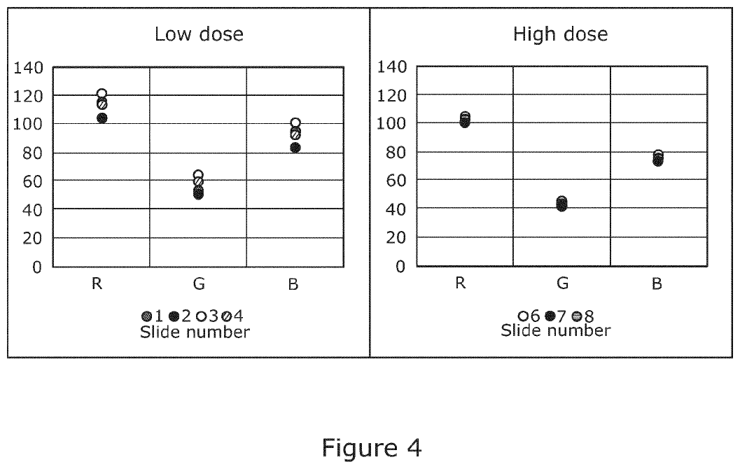

FIG. 4 illustrates the variability of RGB values with cellulose films comprising high doses of chitosan (5% by weight of the film) and with cellulose films comprising low doses of chitosan (2.5% by weight of the film) after staining with hematoxylin for 2 minutes and eosin for 2 minutes.

The films were scanned in the same AT2 scanner and analyzed on QuPath by measuring RGB and L*a*b* values of the film.

As shown in FIG. 4, a high dose of chitosan in the cellulose film reduces the variability of the RGB values compared to cellulose films containing a low dose of chitosan. The high-dose chitosan films also appeared to show a preferential uptake of eosin compared to the lower-dose films. Thus, increasing the levels of chitosan in a film reduces the variability of the imaging process.

1. A quality control device for histopathology staining comprising a cellulose film containing less than two plasticizers.

2. The quality control device according to claim 1, wherein the cellulose film is free from plasticizers.

3. The quality control device according to claim 1, wherein the cellulose film comprises one or more reference articles.

4. The quality control device according to claim 3, wherein the one or more reference articles comprise polymethyl methacrylate (PMMA) particles, silica particles, cellulose fibers, air bubbles, and/or proteins.

5. The quality control device according to claim 1, wherein the cellulose film comprises chitosan, keratin, and/or gelatin.

6. The quality control device according to claim 1, wherein the cellulose film comprises a coating on at least one side thereof.

7. The quality control device according to claim 1, wherein the cellulose film is between 10 and 50 μm thick, preferably between 20 and 30 μm thick.

8. The quality control device according to claim 1, wherein the cellulose film is adhered to a protective sheet.

9. The quality control device according to claim 1, further comprising an adhesive on one side thereof.

10. The quality control device according to claim 9, further comprising a barrier coating on the side of the cellulose film that comprises the adhesive.

11. A surface comprising the quality control device according to claim 1 adhered thereto.

12. A use of a cellulose film containing less than two plasticisers-plasticizers, wherein the cellulose film is used in a quality control device for histopathology staining.

13. The quality control device according to claim 1, wherein the device is used for correcting image variation in a histopathological staining and imaging process.

14. A method of correcting image variation arising from a process of staining and imaging a tissue sample, the method comprising:
staining a quality control device according to claim 1 and the tissue sample under the same conditions;
imaging the quality control device;
quantifying the staining and imaging variation in the quality control device image;
imaging the tissue sample; and
correcting the tissue sample image using the quantification of the staining and imaging variation in the quality control device image.
